(19) United States
(12) Patent Application Publication
(10) Pub. No.: US 2004/0236281 A1
(43) Pub. Date: Nov. 25, 2004

(54) Single-use syringe

(76) Inventor: Frank Popovsky, New South Wales (AU)

Correspondence Address: Testa Hurwitz & Thibeault, Patent Administrator, High Street Tower, 125 High Street, Boston, MA, 02110, US

(21) Appl. No.: 10/484,985

(22) PCT Filed: Oct. 30, 2001
(86) PCT No.: PCT/AU01/01391

(30) Foreign Application Priority Data
Jul. 26, 2001 (AU) PR6575

(51) Int. Cl. A61M 5/00

(52) U.S. Cl. 604/110

(57) ABSTRACT

A single-use syringe includes a shield (2), a barrel (1) mounted for axial movement within the shield and being biased to a rearward position by resilient biasing means (10), and a plunger assembly (3) mounted for axial movement within the barrel. A resilient locking member (6) initially prevents the barrel from moving rearwardly. When fluid is expressed from the barrel, the barrel moves forward thereby disabling rearward movement prevention means (15) and enabling forward movement prevention means (14) such that the barrel can move rearwardly and is locked in the rearward position to prevent repeated use.

### FIELD OF THE INVENTION

The invention relates to a single-use syringe.

### BACKGROUND ART

It is known to employ a single-use syringe to reduce the risk of needle-sharing amongst drug addicts, and to reduce the risk of needle-stick injuries amongst health-care workers and the like. Many different designs of single-use syringes have been suggested. The present invention relates to an alternative design which offers substantial advantages over known designs.

### SUMMARY OF INVENTION

The present invention provides a single-use syringe according to the following claims. Preferred features of the invention will be apparent from the dependant claims and from the following description of the preferred embodiment.

### BRIEF DESCRIPTION OF DRAWINGS

The invention will now be described in a non-limiting manner with respect to a preferred embodiment in which:—

FIGS. 1 to 3 are sequential, longitudinal, sectional and cutaway views of a preferred embodiment of a single-use syringe according to the present invention;

FIGS. 4 and 5 are expanded views of FIGS. 2 and 3, respectively, showing detail of the resilient locking member; and

FIG. 6 is a perspective view of the resilient locking member.

### DETAILED DESCRIPTION OF PREFERRED EMBODIMENT

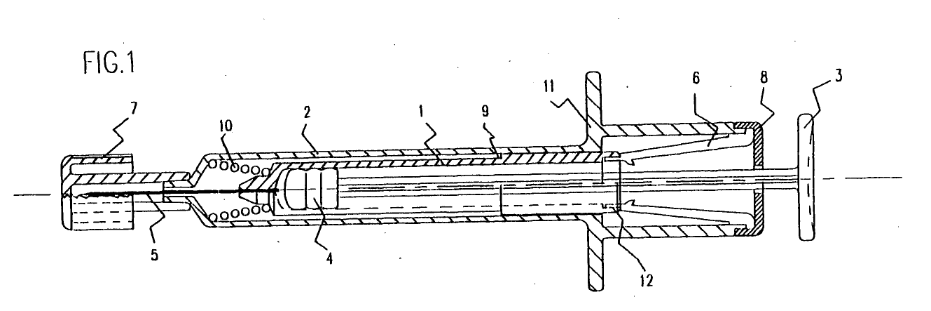

With reference to FIG. 1 there is illustrated a single-use syringe comprising a shield 2, a barrel 1 mounted for axial reciprocation within the shield 2, and a plunger 4 mounted for axial reciprocation within the barrel 1.

Resilient biasing means 10, in the form of a compression spring, is disposed between the interior of the forward end of shield 2 and the exterior of the forward end of barrel 1 such that the resilient biasing means 10 tends to bias the barrel 1 rearwardly with respect to the shield 2.

As is conventional, a needle 5 is mounted at the forward end of barrel 1 and, prior to use, the needle is covered by a cap 7.

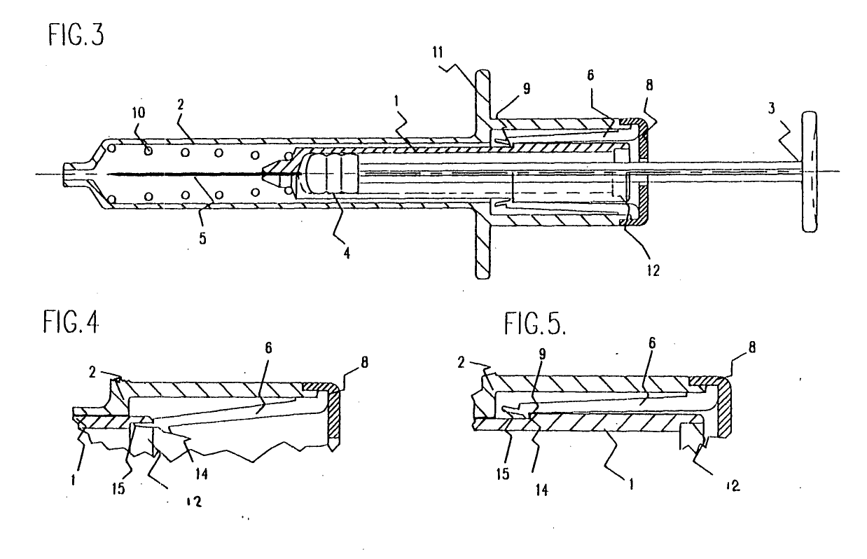

Within a cavity defined at the rearward end of shield 2 is a resilient locking member 6, the shape of which will be best understood with reference to FIG. 6.

Resilient locking member 6 consists of a pair of circumferentially-opposed, forwardly-extending resilient fingers which extend from a ring-shaped base which is mounted relative to the shield 2.

Each of the forwardly-extending resilient fingers terminates in a rearward movement prevention means 15 and a forward movement prevention means 14 in the form of an inwardly-directed hook.

With further reference to FIG. 1, the ring-shaped base of the resilient locking member 6 is mounted relative to shield 2 and is located between the body of the shield 2 and a closure cap 8 for the shield. The closure cap 8 is provided to facilitate assembly of the syringe which will be described further hereunder. After assembly, closure cap 8 is ultrasonically or thermo welded to shield 2.

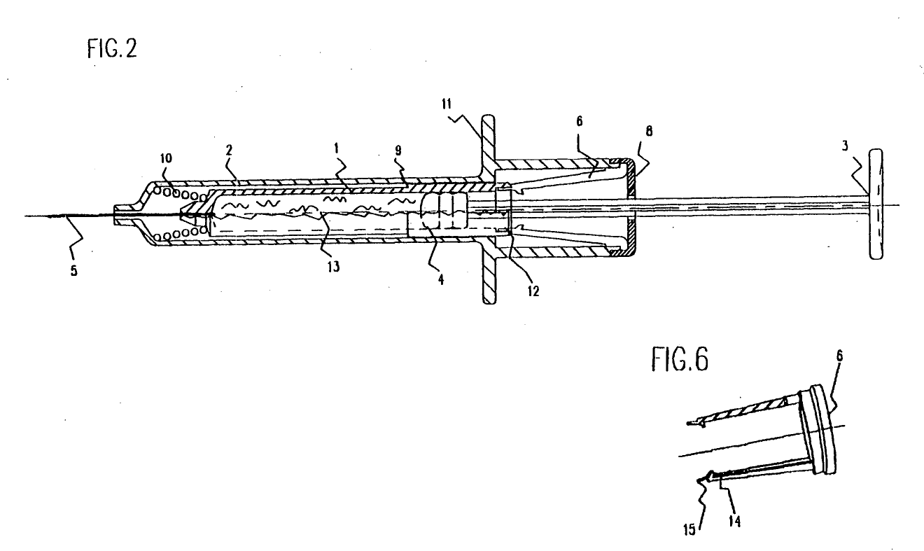

The rearward end of the barrel 1 includes a rearwardly-facing shoulder 12 disposed on the interior of the barrel 1 which may be engaged by the rearward movement prevention means 15. Alternatively, the rearward prevention means 15 includes a forwardly-facing shoulder which engages the rearmost extent of the barrel 1. As illustrated in FIG. 4, the engagement occurs between the forwardly-facing shoulder of the rearward movement prevention means 15 and the rearmost extent of the barrel 1.

The generally cylindrical exterior of the barrel 1 includes a forwardly-facing shoulder 9 which can be engaged by the forward movement prevention means 14 as will be described in more detail hereunder.

With further reference to FIG. 1 and FIG. 4, it will be noted that the resilient biasing means 10 initially fails to urge the barrel 1 in a rearward direction with respect to shield 2 by virtue of the presence of rearward movement prevention means 15 engaging the rear of the barrel 1. This may be best understood with reference to the detailed illustration in FIG. 4.

In use, the cap 7 is first removed to expose needle 5 and thereafter the fluid is aspirated into the interior of the barrel 1 as shown in FIG. 2 by withdrawing the plunger assembly 3, and more particularly the rubber plunger 4, relative to the barrel 1. It will be understood that rearward movement of the plunger assembly 3, and particularly the rubber plunger 4, within the barrel 1 causes a frictional force between the plunger 4 and the interior of barrel 1 which tends to urge the barrel 1 in the rearward direction with respect to shield 2. However, rearward movement of the barrel 1 with respect to shield 2 continues to be prevented by the engagement of rearward movement prevention means 15 with the rear of the barrel 1 as best shown in FIG. 4.

At this point it is worth noting that, whilst the preferred embodiment is described with reference to a syringe which is provided to the user without a fluid already present in the interior of the barrel 1, it is possible that the syringe could be provided to the user in the state shown in FIG. 2. Put differently, the syringe could be provided in a pre-filled configuration as shown in FIG. 2.

With further reference to FIG. 2 and FIG. 4, it will be appreciated that as soon as the barrel 1 moves forwardly with respect to shield 2 thereby compressing resilient biasing means 10, the rearward movement prevention means 15 will disengage from the rear of the barrel 1 and it is important to note at this stage that the forwardly-projecting fingers of the resilient locking member 6 are resilient and tend to spring outwardly so that, as the barrel 1 subsequently travels rearwardly with respect to shield 2, the rearward movement prevention means 15 are not re-engaged with the rear of the barrel 1. Put differently, with comparative reference between FIG. 2 and FIG. 3 (or with comparative reference to FIG. 4 and FIG. 5), the forwardly-projecting fingers of the resilient locking member 6 initially adopt a partially frusto-conical configuration as shown in FIGS. 2 and 4. However, when the barrel 1 moves forward and the fingers are disengaged from the rear of barrel 1, they spring outwardly to the partially cylindrical configuration shown in FIGS. 3 and 5.

It will be understood that the act of expressing fluid from the interior of the barrel 1 causes the barrel 1 to move forwardly with respect to shield 2 thereby compressing resilient biasing means 10. In this regard, it will be noted with reference to FIG. 1 or FIG. 2 that the syringe is provided to the user with the barrel not quite in the fully forward position, but closely adjacent thereto so that only a small forward movement of the barrel 1 occurs during initiation of the injection.

The act of injecting the fluid into the patient (ie the squeezing together of the thumb-engaging portion of the plunger assembly 3 and finger-engaging portions 11 of the shield 2) causes barrel 1 to move to the fully forward position which results in the disengagement and disablement of the rearward movement prevention means 15. As soon as the injection is completed and the squeezing force applied by the user to the thumb-engaging portion of the plunger assembly 3 and finger-engaging portions 11 on the shield 2 is complete, the resilient biasing means 10 urges the barrel 1 rearwardly with respect to the shield 2 to the fully rearward configuration shown in FIG. 3 whereat it is locked by the forward movement prevention means 14 as will now be described.

With reference to FIGS. 3 and 5, it will be noted that the forwardly-projecting fingers of the resilient locking member 6, and more particularly the forward movement prevention means 14, have engaged the forward-facing shoulder 9 which is formed on the exterior of the barrel 1. This inter-engagement between the forward movement prevention means 14 and forward-facing shoulder 9 prevents subsequent forward movement of the barrel 1 relative to the shield 2 and hence prevents a subsequent exposure of the needle 5 and repeat usage of the single-use syringe.

It will be understood that the resilient fingers of the resilient locking member 6 adopt a neutral or natural position when they are not engaged with either the rear of the barrel 1 or the forwardly-facing shoulder 9 on the exterior of the barrel. This natural position will be one in which the fingers are sufficiently spread apart so that the rearward movement prevention means 15 do not engage the rear of the barrel 1, but not so spread apart such that the forward movement prevention means 14 fail to engage the forwardly-facing shoulder 9 on the exterior of the barrel 1.

In summary, squeezing together of the thumb-engaging portion of the plunger assembly 3 and finger-engaging portions 11 during the act of injection causes a small forward movement of the barrel 1 relative to the shield 2 which disengages the fingers of the resilient locking member 6 from the rear of the barrel 1. After completion of the injection, the barrel 1 is biased to the fully rearward position by the compression spring whereat the needle 5 is shielded and whereat the barrel 1 is locked by the fingers of the resilient locking member 6, and particularly the forward movement prevention means 14, engaging the forwardly-facing shoulder 9 on the exterior of the barrel 1.

Manufacture of the single-use syringe according to the present invention is very simple in that first the resilient biasing means 10 and then the needle 5, barrel 1 and plunger assembly 3 are inserted into the shield 2 from the rear thereof. Thereafter, the resilient locking member 6 is located as shown in FIG. 1 with the rearward movement prevention means 15 engaging the rear of the barrel 1 as shown. Finally, the closure cap 8 is ultrasonically or thermo welded to the shield 2.

It will be appreciated that the configuration of the preferred embodiment is particularly advantageous in that the forward movement of the barrel relative to the shield which occurs during injection simultaneously disables the rearward movement prevention means 15 and enables the forward movement prevention means 14, each of which is integrally located on the forwardly-projecting fingers of the resilient locking member 6. As soon as the injection is complete, the resilient biasing means 10 urges the barrel rearwardly with respect to the shield to a position whereat it is locked to prevent subsequent use.

It is of course important to ensure that forward movement of the barrel 1 relative to the shield 2 does not occur inadvertently prior to the injection taking place. For example, it is important that forward movement of the barrel 1 relative to the shield 2 does not occur during transportation, or preliminary handling of the syringe. For this purpose, a removable spacer may be inserted between the rear of the closure cap 8 and the thumb engaging portion of the plunger assembly 3. Alternatively, the plunger rod may be shortened such that the thumb-engaging portion of the plunger assembly 3 abuts the rear of the closure cap 8 when in the configuration shown in FIG. 1.

It will be appreciated that the present invention has significant ergonomic advantages in that the operation of the syringe is entirely conventional and no extraneous operations are required to lock the device to prevent subsequent use.

1. A single-use syringe including:—
a shield;
a barrel having a needle at its forward end, the barrel being mounted for axial movement within the shield between a forward position whereat the needle is exposed and a rearward position whereat the needle is shielded;
resilient biasing means for biasing the barrel towards the rearward position; and
characterised in that:—
rearward movement prevention means initially prevent rearward movement of the barrel to said rearward position, and
forward movement of the barrel disables said rearward movement prevention means such that, subsequent to said forward movement, said resilient biasing means can bias the barrel to said rearward position; and
forward movement prevention means then prevent subsequent forward movement of the barrel; and
said rearward movement prevention means and said forward movement prevention means are integrally located on a resilient locking member; and
the resilient locking member initially engages the barrel and prevents rearward movement of the barrel and, subsequent to said forward movement of the barrel, the resilient locking member engages the barrel and prevents forward movement of the barrel.

2. A single-use syringe as claimed in claim 1, wherein the resilient locking member is comprised of a plurality of circumferentially spaced, forwardly-directed fingers.

3. A single-use syringe as claimed in claim 2, wherein the resilient locking member is bi-furcated and includes a pair of circumferentially-opposed, forwardly-directed fingers.

4. A single-use syringe as claimed in claim 2, wherein each finger includes an inwardly directed hook.

5. A single-use syringe as claimed in claim 1, wherein the syringe includes a plunger assembly mounted for axial reciprocation within the barrel, the plunger assembly including a thumb-engaging portion and the shield including a finger-engaging portion, and wherein squeezing together of the thumb-engaging portion and finger-engaging portion to express fluid from the interior of the barrel causes said forward movement.

6. A single-use syringe as claimed in claim 1, wherein the syringe is supplied with the barrel adjacent said fully forward position such that the magnitude of said forward movement is small relative to the subsequent rearward movement under the influence of the resilient biasing means.
